(19) United States
(12) Patent Application Publication
(10) Pub. No.: US 2004/0035751 A1
(43) Pub. Date: Feb. 26, 2004

(54) Inorganic mesoporous solids, a process for their preparation and their use, notably as catalysts and adsorbents

(75) Inventor: Dominique Plee, Lons (FR)

Correspondence Address: HUNTON & WILLIAMS LLP, INTELLECTUAL PROPERTY DEPARTMENT, 1900 K STREET, N.W., SUITE 1200, WASHINGTON, DC, 20006-1109, US

(73) Assignee: CECA, S.A., Puteaux (FR)

(21) Appl. No.: 10/437,456

(22) Filed: May 14, 2003

Related U.S. Application Data
(63) Continuation of application No. PCT/FR01/03496, filed on Nov. 9, 2001.

(30) Foreign Application Priority Data
Nov. 14, 2000 (FR) 00/14595

(51) Int. Cl. C04B 38/00; C01B 33/26; C10G 11/00; C07C 5/22; C07C 2/54

(52) U.S. Cl. 208/113; 423/592.1; 423/702; 423/328.1; 501/80; 501/81; 585/446; 585/470; 585/643; 585/743; 585/708; 585/709; 585/671

(57) ABSTRACT

The present invention relates to new mesoporous inorganic solids in the form of primary and/or secondary inorganic particles of D10≧1 μm and D50≧3 μm, preferably from D10≧2 μm and D50≧10 μm the size of which can go up to 10 mm, wherein the microporous volume (pores of size less than or equal to 2 μm) represents at most 10% of the total porous volume up to 300 nm.

These solids can advantageously be used as catalytic component supports in polymerization reactions, as reaction catalysts in the refining and petrochemical fields, as adsorbents for separating the components of a gaseous or liquid mixture consisting of at least 2 different compounds and as chromatography column packing supports.

### CROSS REFERENCE TO RELATED APPLICATIONS

The present application is a continuation of PCT/FR01/03496, filed Nov. 19, 2001, which claims the benefit of French Patent Application No. 00/14595, filed Nov. 14, 2000, the disclosures of both which are hereby incorporated by reference.

### BACKGROUND OF THE INVENTION:

1. Field of the Invention

This invention relates to a process for the preparation of a new family of mesoporous inorganic particles; the process permits precision control of the granulometric and morphological distribution of the prepared particles which can advantageously be used as catalyst supports, as catalysts and /or for the separation of gaseous phase compounds having different boiling points such as for the packing of chromatography columns.

2. Description of the Related Art

The mesoporous particles have a significant industrial utility, not only both as catalysts and catalyst supports but also as adsorbents, insofar as their significant porosity, expressed in terms of the surface to volume ratio, permits the molecules that they are put into contact with to have easy access to the heart of the particles and to react on a significant surface, magnifying in this way the catalytic and/or adsorbent properties of these materials.

The synthesis of mesoporous inorganic solids, having a narrow and calibrated mesopore distribution by surface active agent structuring effect, was first described by Sylvania Electric Products in U.S. Pat. No. 3.556.725.

During the course of the 1990's, Mobil Corporation undertook numerous efforts relating to mesoporous inorganic solids, particularly with (alumino)silicic compounds and more particularly the compound MCM 41 (Mobil Composition Of Matter 41) whose synthesis process is described in Nature, 1992, vol 359, pp.710-712 and which is the object of numerous patents and scientific articles; such mesoporous materials are now well known at the laboratory scale at the level of their structure and of their porous distribution, the synthesis conditions as well as possible applications as a catalyst and/or as an adsorbent.

It is thus known how to prepare such inorganic mesoporous organized solids, having a narrow pore size distribution up to the range of 2 to 10 nanometers. For instance MOBIL U.S. Pat. No. 5,057,296 describes a method for preparing a composition of matter comprising a crystalline, non-lamellar inorganic phase, having, after calcination, an arrangement of pores of uniform size equal to at least 1.3 nm, with at least an X-ray diffraction peak corresponding to a reticular distance greater than 1.8 nm and having a benzene adsorption capacity greater than 15% by weight at 25° C. and 50 torrs starting from an HiSil type silica in admixture with a solution de tetramethylammonium silicate.

Other work has shown the influence of pH on the size and the morphology of the synthesized mesoporous solid particles: in acid medium and for a molar ratio of HCl/silica equal to 2.05, the synthesized particles have a size of 12 to 13 μm and adopt a spiral form (DI RENZO F. et al , Microporous and Mesoporous Materials , 28 ( 1999 ) , p. 437- 446 ); in neutral medium, the size of the particles decreases and is not more than about 3 μm and their morphology depends on the ionic force; finally, in basic media, the size of the particles is only on the μm or submicronic order.

In a general manner, the syntheses de silicic mesoporous solids are carried out starting from tetraethylortho silicate (TEOS), tetraalkylammonium or sodium silicate or from precipitated silicate.

TEOS gives rise to the disadvantage, besides being a costly reactant, of generating ethanol at the time of the hydrolysis. But, used in a non-basic medium, it is the only source de silica that permits preparation of mesoporous solid particles of a few μm. Another disadvantage of the syntheses of mesoporous solids in neutral or acid media relates to the yield of surface active agent expressed as the ratio between the surface active introduced at the beginning of the synthesis and the surface active retained in the formed solid which is definitely less than 100%.

The use of silicates, of lower cost, is limited to basic pHs that permit obtaining of particles of very small size, typically less than a micrometer but of very irregular morphology. On the other hand, the yield of surface active agent is 100%.

The preparation of particles of mesoporous solids of a size greater than 15-20 μm requires a supplemental stage of agglomeration of the primary particles obtained according to one or the other of the processes recalled hereinbelow.

Among the agglomeration processes well known to a person skilled in the art, there can be cited:

extrusion in which a paste composed of primary particles of mesoporous solid, a binder, a liquid and possibly an extrusion additive are made to pass across a die and then small rods or an extrudate that is cut at a chosen length is recovered.

agglomeration on granulator tray of the same ingredients as for the extrusion in order to form pallets, in effect a snowball,

compacting under pressure of a mixture of primary particles of mesoporous solid, a binder and possibly a small amount of liquid under pressure so as to obtain the desired cohesion.

atomization for the particles of smallest size.

Now, these agglomeration processes have the disadvantages listed hereinbelow:

the extrusion product of the particles of identical diameter but of variable length, which can have a disastrous influence on the diffusional properties of the material; in addition this technique is well adapted for diameters greater than 500 μm but less adapted for lower diameters;

the granulation product of the particles under the form of pellets, therefore rather spherical, with a large size distribution, which for certain applications, can constitute a handicap. The only means with this technique to obtain particles with a narrow granulometric distribution is to operate granulometric selections subsequent to the actual granulation stage to the detriment of the yield and/or the productivity. In addition, granulation is a technique that is quite adapted for particle sizes greater than a millimeter;

the compacting is especially useful for the formulation of pharmaceutical products and involves particles of even greater sizes: a few mm at least;

atomization permits manufacture of particles of about 20 to 200 μm with a narrow particle size distribution. However this technique does not permit obtaining of secondary particles having mechanical properties sufficient for the majority of the envisioned uses (catalysis, adsorption), particularly when the source of silica is TEOS.

All of these considerations show that there exists a real need to propose un system permitting the obtaining of mesoporous solid particles in a range of particle sizes including between 1 μm and a few mm and not having the disadvantages previously discussed. To demonstrate that it is very important to be able to regulate the granulometric distribution of the mesoporous solids formed, it is sufficient to cite the example of the polymerization catalysis of the olefins: it is well known, that the size of the silica particles used as a support in polymerization catalysis plays a very important role in the morphological control of the polymer product. Certain work in this way the advantage of the MCM 41 support in the polymerization of propylene; the synthesis of isotactic polypropylene having high melting points was demonstrated on the System MCM 41/MAO/Zr Cl₂ ( EBI ) [with EBI=1,2-bis(inden -1-yl)-ethane] (<<Stereospecific propene polymerization catalysis using an organometallic modified mesoporous silicate>>, TUDOR, J. and O'HARE, D. , Chem. Commun.,1997, p.603-604)

### SUMMARY OF THE INVENTION:

The present invention relates to mesoporous inorganic solids existing in the form of primary and/or secondary inorganic particles of D10≧1 μm and D50≧3 μm, preferably from D10≧2 μm and D50≧10 μm, the size of which can go up to 10 mm, preferably up to 3 mm and advantageously up to 1.5 mm, of the total composition corresponding to the formula:

M_{n/q}(WₐX_{b}Y_{c}Z_{d}Oₕ)

wherein M represents one or several ions , such as the ammonium ion, Group IA IIA and VIIB ions, and notably the hydrogen and/or sodium ions, n and q represent respectively the equivalent fraction and the valence of the ion(s) M and n/q represents le number of moles or the molar fraction of the ion(s) M,

W represents one or several divalent elements, such as manganese, cobalt, iron and/or magnesium,

X represents one or several trivalent elements, such as aluminum, boron, iron and/or gallium,

Y represents one or several tetravalent elements, such as silicon and/or germanium, and preferably silicon

Z represents one or several pentavalent elements, such as phosphorus,

O represents oxygen,

a, b, c and d are the respective molar fractions of W, X, Y and Z with a+b+c+d =1 and 1≦h≦2,5,

wherein the microporous volume (pores of size less than or equal to 2 μm) represents at most 10% of the total porous volume corresponding to pores of size going up to 300 nm,

and

wherein either the mesoporous volume corresponding to the pores of size going from 2 to 10 nm is greater than or equal to 0.18 cm³/g, and preferably greater than or equal to 0.3 cm³/g, wherein the diameter of the maximum distribution peak DFT (Dmax) is such that 2≦Dmax≦10 nm, preferably 2≦Dmax≦5 nm, and wherein the porous volume corresponding to the pores of size Dmax±15% represents at least 70%, preferably at least 80% et advantageously 90% of the porous volume corresponding to the pores of size ranging between 2 and 10 nm,

or wherein the mesoporous volume corresponding to pores going from 4 to 15 nm is greater than or equal to 0.7 cm³/g, and preferably greater than or equal to 1 cm³/g, wherein the diameter of the maximum distribution peak DFT (Dmax) includes ranges in a larger sense between 4 and 15 nm and wherein the porous volume corresponding to the pores of size Dmax±20% represents at least 45%, preferably at least 50% of the porous volume corresponding to the pores of size ranging between 4 and 15 nm.

The porous volumes are measured by N₂ adsorption at 77 K.

The porous volumes corresponding to pores having a size greater than or equal to 2 nm and less than or equal to 300 nm are measured par la DFT method (cylindrical pores).

The porous volume corresponding to pores of a size less than or equal to 2 nm (microporous volume according to IUPAC) are measured by the t-plot method.

D10, D50 and D90 represent the diameters of the particles below in which 10%, 50% and 90% by weight of the particles are found, respectively, the D 50 giving a good approximation of the size of the particles.

Among the inorganic solids according to the invention, the preferred ones are those having a chemical composition represented empirically by the formula:

M_{n/q}(X_{b}Y_{c}Oₕ)

with X=Al , Y=Si and possibly Ti, b+c=1 and 0≦b≦1,

and advantageously the silicas.

The invention equally relates to a process for manufacturing the inorganic solids described above comprising the following steps:

contacting and reacting a reaction mixture containing

a solid inorganic source in the form of primary and/or secondary particles from D10≧1 μm and D50≧3 μm, preferably from D10≧2 μm and D50≧10 μm wherein the size can go up to 10 mm, of total composition corresponding to the formula:

M_{n/q}(WₐX_{b}Y_{c}Z_{d}Oₕ)

where M, W, X, Y, Z, n, q, a, b, c, d and h have the same meaning as previously,

a mobilizing agent of the solid inorganic source,

un pore calibrating agent, for example a surface active agent,

and a solvent, preferably water,

optionally in the presence of an inflating agent that is soluble in micelles, preferably trimethylbenzene,

then filtration, washing, drying and optionally elimination of the pore calibrating agent and calcination of the inorganic particles obtained, characterized in that the conditions of temperature, agitation and of reaction length are such that no notable modification of the morphology and of the size of the particles present in the course of said reaction is observed. as can be appreciated by electronic scan microscopy (ESM)and laser granulometry.

### DETAILED DESCRIPTION OF PREFERRED EMBODIMENTS:

By way of examples of pore calibrating agents, particular reference can be made to the surface active agents containing ammonium or quaternary phosphonium ions, substituted by aryl or alkyl groups having from 6 to 36 identical or different carbon atoms, in association with hydroxide, halide or silicate anions and notably those that contain cetyltrimethylammonium, cetyltrimethylphosphonium, octadecyltrimethylammonium, octadecyltrimethylphosphonium, benzyltrimethylammonium, cetylpyridinium, decyltrimethylammonium, dimethyldidodecylammonium, trimethyldodecylammonium ions as well as amines such as dodecylamine and hexadecylamine.

The solvent can be organic but is preferably aqueous.

By way of example of oxide mobilizing agents, mention can be made of organic and mineral bases, with soda being particularly preferred.

The pH of the reaction mixture is generally not critical and may vary between 1 and 14. The crystallization of the solid can be carried out with or without agitation, so long as it is sufficiently moderate so as to not cause attrition of the particles present and therefore an increase in the proportion of fine particles. The crystallization temperature generally ranges between ambient temperature and 200° C. and the duration of the crystallization reaction can generally go from a few minutes to a few days.

The duration of the reaction stage is controlled and optimized by ESM and laser granulometry, too long of a reaction duration risking an increase in the proportion of fine particles.

At the outlet of the actual reaction stage, a solid suspended in the solvent is obtained that is filtered, washed and dried; the product obtained having, after calcination intended in particular to eliminate the surface active agent by combustion, in the form of inorganic solid particles possessing pores or regular size that may be of cubic or hexagonal symmetry according to the conditions of synthesis. In the case of hexagonal symmetry, the pores are all parallel.

The process according to the invention in particular gives rise to the following advantages:

1. It is possible to influence the morphology of the mesoporous oxide particles through the morphology of the particles of the oxide source. it is therefore possible, owing to the process according to the invention to optimize the morphology of the mesoporous oxide particles according to desired characteristics such as for example the flow or the ability of these particles to not accumulate electrostatic charges.

2. It is possible to influence the size of the mesoporous oxide particles by modifying the size of the particles of the oxide source. In this way, by increasing or by decreasing the size of the oxide source particles, it is possible to increase or decrease the size of the mesoporous oxide particles.

3. It is possible to influence the granulometric distribution of the mesoporous oxide particles by adjusting the granulometric distribution of the particles of the oxide source. In effect the size distribution of the mesoporous oxide particles, measured by laser granulometry, is roughly related to the granulometric distribution of the oxide source particles employed. The process according to the invention is of particular interest when looking for narrow particle size distributions; in this case, it is advisable to employ an oxide source having a narrow particle size distribution.

4. According to the operating conditions used, it is possible to have the distances between the pores or the thicknesses of the walls between the pores vary. For example, the pH permits varying the thickness of the walls: an interpretation currently admitted by numerous authors is that in a basic medium, silica arranges itself around the micelles of the surface active agent through interaction between the cationic head of the surface active agent and the ionized silanol groups that are found on the silica surface.

5. It is possible to vary the pore size distribution of the particles formed with or without addition, more or less, of an inflating agent: when in the course of the synthesis of the particles according to the invention an inflating agent is employed, solids having large pores are obtained, namely, the mesoporous solids according to the invention wherein

B-1 the mesoporous volume corresponding to pores ranging from 4 to 15 nm is greater than or equal to 0.7 cm³/g, and preferably greater or equal to 1 cm³/g,

B-2 the maximum distribution peak diameter DFT (Dmax) ranges between 4 and 15 nm

and the porous volume corresponding to the pores of size Dmax±20% represents at least 45% preferably at least 50% of the porous volume corresponding to the pores of size ranging between 4 and 15 nm.

It has been observed that the concentration of the inflating agent influences the size of the pores: the more the concentration of the inflating agent is raised, the greater is the size of the pores.

When no inflating agent is employed, solids according to the invention are obtained wherein:

A-1 the mesoporous volume corresponding to the pores of size ranging from 2 to 10 nm is greater than or equal to 0.18 cm³/g, and preferably greater than or equal to 0.3 cm³/g,

A-2 the maximum distribution peak diameter DFT (Dmax) is such that 2 nm≦Dmax≦10 nm, preferably 2 nm≦Dmax≦5 nm

and A-3 the porous volume corresponding to the pores of size Dmax±15% represents at least 70% preferably at least 80% and advantageously 90% of the porous volume corresponding to the pores of size ranging between 2 and 10 nm,

The particles according to the invention of D50≧10 μm can advantageously serve as catalytic component supports (for this reason, they can be called “support particles” in what follows) for the polymerization of various polymers notably polyamides, polyesters, olefins and styrenic compounds, jointly named olefins in what follows, etc.; by olefins is understood here the polymers resulting from one or several monomers selected among the C₂-C₁₀ olefins, vinylic monomers such as vinyl acetate and aromatic vinylic monomers such as styrene and its derivatives.

A catalytic component for the polymerization of the olefins can be obtained by the association of a transition metal compound with the support particles. That transition metal can be titanium, zirconium, hafnium, chromium, vanadium or any other metal capable under conditions suited for catalyzing the polymerization of the olefins. For example, a solid catalytic component can be obtained by association with the support, of a titanium compound, of chlorine, possibly of an aluminum compound, possibly an electron acceptor or donor as well as any other compound usable in solid components of Ziegler-Natta type or metallocene.

Some polymers (notably copolymers and prepolymers) can be obtained by polymerization of monomer(s), in the presence of the catalytic component according to the invention by processes in suspension, in solution, in gaseous phase or en masse.

The particles according to the invention can equally serve as catalysts in reactions in the field of petrochemical refining, typically alkylation, isomerization, dismutation, cracking reactions, which are in general reactions acid nature.

The particles according to the invention can equally serve as adsorbents for separating the components of a gaseous or liquid mixture comprising at least 2 different compounds in an adsorption process. From a practical point of view, the preferred adsorbents are those wherein the granulometry is in general at least on the order of a millimeter. The particles according to the invention can be used wherein the granulometry corresponds to that desired, or it may well be necessary, if their granulometry is insufficient, to agglomerate them before their employment for example according to one and/or another of the agglomeration techniques set out above (extrusion, agglomeration, compacting and atomization)

By way of example of adsorption processes, reference will be made more particularly to those functioning in a cyclic manner, which include the following stages functioning alternatively which are detailed hereinbelow:

a/ having said mixture pass in an adsorption zone containing the mesoporous particles and recovering either the least absorbed compound(s) or a gaseous mixture enriched in the least adsorbed compound(s) at the exit of the adsorption zone,

b/ desorbing the adsorbed compound(s) in the adsorption zone and regenerating the adsorption zone in a manner so as to restore its adsorption capacity to it.

The desorption/regeneration stage b/ is carried out by means of vacuum (aspiration), by purging of the adsorption zone with one or several inert gases and/or with a part of the gaseous flux obtained at the exit of the adsorption zone, by increasing temperature or by combination of the regenerations by aspiration, by purging and/or temperature variation.

Applicant's preferred processes are of PSA or VSA type, de TSA type or of a combination of these different types of processes (PTSA).

This process is particularly well suited for the separation of VOC present even at very low concentration in the gaseous flux preferably based on dry or humid air.

The process of the present invention is equally well suited for the purification of hydrocarbons particularly of oxygenated hydrocarbons and still more specifically of hydrocarbons belonging to the group of ketones, aldehydes, acids or alcohols, in admixture with compounds, preferably in impure or trace state.

Among the particles according to the invention, those with 1≦D10≦3 μm and 3≦D50≦15 μm, preferably those of silica base, can advantageously be used for the packing of chromatography columns. By way of example, in preparative chromatography it is preferred to use particles of D50 close to 12 μm and in HPLC (high performance liquid phase chromatography) it is preferred to use particles of D50 close to 5 μm.

### EXAMPLE 1

1)In a cylindrical reactor of 1 l and of 8 cm of diameter, a solution containing 310 ml of water, 8.3 g of soda and 29.4 g de NORAMIUM® MS 50 sold by CECA (trimethylalkylammonium chloride with an alkyl chain length of 16 to 18 carbon atoms) is prepared.

2) Still at ambient temperature, there is added under agitation by anchor or by blades 33 g (counted in anhydride equivalents) precipitated pulverulent silica sold by CECA under the name LEVILITE® wherein the pore size distribution is large and ranges toward 20 nm and wherein certain of the characteristics are collected in the table 1 below.

The typical suspension composition is:

0.19 Na₂O—0.084 C16⁺—SiO₂—32 H₂O

2) Still under agitation, the reaction medium is taken to 100° C., the temperature being maintained for 3 h. The solid is filtered then washed with 3 l of water and dried in a ventilated drying oven at 70° C. and calcined at 550° C. by going up in 5 h from 25° C. to 550° C. then at that level for 1 h.

After synthesis, the solid is characterized by adsorption/desorption of N₂ at 77 K (ASAP 2010 of MICROMERITICS ) and by the granulometer LASER (MALVERN ) The pore size distribution is calculated according to the method DFT.

One observes on the isotherm the sudden adsorption jump toward P/Ps=0.37, corresponding to the characteristic capillary condensation in the mesopores. Besides, the adsorption capacity of toluene in gaseous phase is measured at 25° C. under a relative pressure of 0.5 , which is equal to 65% by weight.

The characteristics of the starting silica and of the solid obtained are collected in the table 1.

In view of the table 1, it is observed that the granulometric distribution of the mesoporous solid formed and that of the starting silica are practically superimposable with, each other, in particular no fine particles (0% of particles of size less than 2 μm). The synthesis such as it is practiced permits conservation of the morphology of the starting material.

### EXAMPLE 2

The synthesis of example 1 is reproduced with the exception of the movable agitation which is replaced by a magnetic agitation by means of a magnetized bar of 3 cm diameter turning at 100 trs / min.

The solid resulting from this synthesis presents practically the same surface characteristics and porosity as that of example 1 but reveals in the LASER granulometer the existence of fine particles estimated at 4-5% by weight less than 2 μm ; the use of a shearing agitation system favors the abrasion of the particles.

### EXAMPLE 3

The synthesis of example 1 is reproduced by replacing the LEVILITE® with a silica sold by GRACE under the name SYLOPOL® 2104; this silica having a narrow particle size distribution without fine particles (0% of particles less than 15 μm) and a large pore size distribution centering on about 20 to 40 nm.

The characteristics of the starting silica and of the product resulting from the synthesis are indicated in table 1.

The mesoporous solid formed has a sudden adsorption jump of nitrogen for P/Ps=0.37, corresponding to the capillary condensation in the mesopores.

In addition, starting from the results of table 1, it is observed that the granulometric distribution of the solid resulting from the synthesis can be somewhat confused with that of the initial silica with the exception of a little trail towards the particles of small size. The solid formed contains a second porous volume (porous volume 10-300 nm) residue of the synthesis of the starting product.

### EXAMPLE 4

The synthesis of example 1 is reproduced, by using as silica source ZEOSIL® 175 MP sold by RHODIA wherein the pore size distribution is large and situated in the macropores (>50 nm) The granulometric distribution of this silica shows a principal peak towards 150 μm with a large trail towards the particles of lowest granulometry but not any fine particles (0% of particles of size less than 4 μm) The characteristics of the starting silica and of the synthesized mesoporous solid are collected in table 1.

In view of table 1, it is observed that the synthesized solid, even if it does not possess the same median diameter (D50) as the starting silica, reproduces rather faithfully its total distribution. It is also observed that the ratio of the porous volume (2-10 nm) to the porous volume (10-300nm) is less than that of the solid formed in example 1, which can be attributed to porosity residue of the initial silica.

The sudden jump of nitrogen adsorption is observed for P/Ps=0.36, corresponding to the capillary condensation in the mesopores.

The position of the maximum peak of granulometry of the starting silica corresponds to 150 μm whereas that of the solid formed is situated at 90 μm with, for the 2 solids, a large trail towards the small sizes of particles and no fine particles (0%<4 μm).

### EXAMPLE 5

The synthesis of example 1 was reproduced by using as a silica source SYLIPOL® 2104 and a ratio Na₂O over silica of 0.08 instead of 0.19.

The characteristics of the starting silica and of the product resulting from the synthesis are indicated in the table 1 hereinbelow.

The synthesized mesoporous solid is of worse quality that those of the previous examples because of the lower basicity of the medium which permits only one partial transformation of the solid. The sudden jump in nitrogen adsorption is observed for P/Ps=0.36, corresponding to the capillary condensation in the mesopores.

It reproduces rather accurately the granulometric profile of the starting silica.

### EXAMPLE 6

The synthesis of a solid of mesoporous type having larger pores was carried out under the following conditions:

1) A solution containing 300 ml of water, 8.3 g of soda and 29.4 g of NORAMIUM® MS 50 is prepared

2) There is added 27.7 g of trimethylbenzene (TMB) as an inflating agent under agitation so as to permit the solubilization of this molecule in the surface active agent micelle.

3) After agitation for about 15 min, 33 g of SYLIPOL® 2104 (counted by anhydrous equivalent) are added under slow agitation.

4) Rising to 100° C. under agitation and maintain 16 h at this temperature

5) Filtration and washing with 3 l of water

6) Drying at 70° C. in a ventilated drying oven.

7) Calcination at 550° C. by rising in 5 hours from 25° C. to 550° C. and maintaining 1 h at that temperature.

The composition of the synthesis medium is as follows:

0.19 Na₂O—0.084 C16 +−0.42 TMB —1 SiO₂—32 H₂O

The solid is as previously characterized and the results are reported in the table 2 hereinbelow. The solid shows a sudden jump in nitrogen adsorption towards 0.75, corresponding to the capillary condensation in the mesopores and its granulometric distribution very accurately reproduces that of the initial silica.

### EXAMPLE 7

The synthesis of example 1 is reproduced, by suing as oxide source a silica-alumina of molar ratio Si/Al=7 sold by KETJEN in the form of de grains crushed and screened beforehand to below 125 μm. The characteristics of the starting silica-alumina and of the product resulting from the synthesis are indicated in the table 1 hereinbelow.

Examination with MEB shows perfect conservation of the size and of the morphology of the particles during the synthesis. However, a slightly different surface aspect of the formed particle is noted (smoother) from that of the starting silica-alumina particles.

### EXAMPLE 8

In 310 g of water, 29.4 g of NORAMIUM® MS 50 then 8.4 g of soda are dissolved. After agitation for dissolving all of the ingredients, there is dispersed in the mixture 33 g (anhydrous equivalent) of precipitated silica marketed by the applicant under the name LEVILITE® the characteristics of which are set forth in table 1, then it is brought to a temperature of about 100° C., and the mixture maintained at such temperature for 16 h under light agitation. The obtained solid is filtered and washed with 6 l of water then dried so as to exhibit, after a thermal treatment for 2 h at 550° C. under air, the characteristics set forth in table 1.

It is observed that the proportion of fine particles having a size less than 4 μm represents 3% of the total weight of the particles whereas it was 0% for the starting silica, which clearly shows a degradation of the particles during the synthesis. At MEB, it is observed that certain particles are damaged or have burst and that some small fragments appeared.

This clearly demonstrates the influence of the duration of the synthesis on the proportion of fine particles.

### EXAMPLE 9

In 310 g of water, 29.4 g of NORAMIUM® MS 50 are dissolved then 8.4 g of soda. After agitation so as to dissolve all of the ingredients, there is dispersed in the medium 31 g (anhydrous equivalent) of precipitated silica marketed by GRACE Corporation under the name SYLOPOL® 2104 having the characteristics that are set forth in table 1, then brought to a temperature of 100° C., the temperature of the mixture being maintained for 40 h under light agitation. The solid obtained is filtered and washed with 6 l of water then dried so as to give rise to, after a thermal treatment for 2 h at 550° C. under air, the characteristics set forth in table 1.

It is observed that the proportion of fine particles having a size less than 4 μm represents 43% of the total weight of the particles whereas it was 0% for the starting silica, which shows a slight degradation of the particles during the synthesis. In this example, it is also observed that the particles are damaged because of the duration of the synthesis.

### EXAMPLE 10

In a reactor equipped with movable agitation of the Archimedes screw type, 400 ml of a solution containing 33.2 g of soda, dissolved beforehand, are introduced. The agitation is started at 200 trs/ min and 117.6 g of NORAMIUM® MS 50, sold by CECA and 102.2 g of 1,3,5 trimethylbenzene (inflating agent) are added. After 5 min of agitation during which time the emulsion forms, 132 g (anhydrous equivalent) of TIXOSIL® 68, silica sold by RHODIA are introduced.

The reaction medium is brought to 100° C. for 3 h while maintaining the agitation then it is filtered and washed with 12 l of water. The product is then dried at 100° C. for 2 h then activated in a drying oven by raising in 1 h to 550° C. and maintaining this temperature for 2 h under a N₂ sweep The solid is thus characterized by its isothermal adsorption/desorption of N₂ at 77 K which permits a deduction in the surface and porosity values.

The isothermal adsorption/desorption of N₂ at 77 K shows that the solid is a mesoporous solid according to the invention, well formed with a pronounced adsorption step and a relatively narrow pore size distribution.

One also proceeds with a measure of the granulometric distribution by means of a MALVERN granulometer

The characteristics of the obtained solid are set forth in table 2. The comparison of the respective granulometries appears in table 2 hereinbelow:

It can be concluded that it is possible to synthesize mesoporous inorganic solids having large pores according to the invention by isomorphic synthesis in a reactor equipped with an agitation means of the Archimedes screw type. It is also observed, that the width of the granulometric distribution is lower in the solid according to the invention than on the starting silica; this here is an advantage in optics, for use in polymerization catalysis especially because the proportion of fine solid particles according to the invention is greatly diminished (D10) as compared to the starting silica while maintaining the proportion of large particles (D90)

TABLE 1

| | | | | | | Porous | Porous | | | |
|---|---|---|---|---|---|---|---|---|---|---|
| | | | | | Surface | Volume | Volume | V(<2 nm)/ | | V |
| Example | | D10 | D50 | D90 | BET | (2-10 nm) | (10-300 nm) | V(≦300 nm) | Dmax | (Dmax ± 15%) |
| N° | SOLID TYPE | (μm) | (μm) | (μm) | (m²/g) | (cm³/g) | (cm³/g) | (%) | (nm) | /V(2-10 nm) |
| 1/8 | LEVILITE ® | 4.1 | 9 | 22 | 627 | 0.35 | 0.38 | 0 | 20 | — |
| 1 | Solid formed (silica) | 4.8 | 11.3 | 20.8 | 1.050 | 0.78 | 0.12 | 0 | 3.2 | 90 |
| 8 | Solid formed (silica) | — | — | — | 1.100 | 0.73 | 0.13 | 3 | 3.3 | 90 |
| 3/5/9 | SYLOPOL ® 2104 | 30 | 46 | 63 | 324 | 0.20 | 1.53 | — | 20-40 | — |
| 3 | Solid formed (silica) | 12.5 | 46 | 59.6 | 1.157 | 0.85 | 0.67 | 0.1 | 3.3 | 81 |
| 5 | Solid formed (silica) | 15 | 46 | 60 | 692 | 0.39 | 0.71 | 0.3 | 3.1 | 76 |
| 9 | Solid formed (silica) | — | — | — | 1.114 | 1.04 | — | — | 3.3 | 90 |
| 4 | ZEOSIL ® 175 MP | 12.6 | 78.2 | 158 | 155 | 0.09 | 0.22 | — | 150 | — |
| 4 | Solid formed (silica) | 13.1 | 62.6 | 106 | 1.126 | 0.80 | 0.38 | 0.1 | 3.3 | 83 |
| 7 | Starting Silica-alumina | 5.1 | 45 | 77 | 403 | 0.35 | 0.11 | — | 10-30 | — |
| 7 | Solid formed | 6.5 | 51 | 90 | 290 | 0.21 | 0.05 | 1 | 3 | 80 |
| | (silica-alumina) | | | | | | | | | |

TABLE 2

| | | | | | | Porous | Porous | | | |
|---|---|---|---|---|---|---|---|---|---|---|
| | | | | | Surface | Volume | Volume | Volume(<2 nm)/ | | V |
| Example | | D10 | D50 | D90 | BET | (4-15 nm) | (15-300 nm) | V(≦300 nm) | Dmax | (Dmax ± 15%)/ |
| N° | Product | (μm) | (μm) | (μm) | (cm²/g) | (cm³/g) | (cm³/g) | (%) | (nm) | V(4-15 nm) |
| 6 | SYLOPOL ® 2104 | 30 | 46 | 63 | 324 | 0.20 | 1.53 | — | 20-40 | — |
| 6 | Solid formed (silica) | 8.2 | 46 | 20.8 | 1.070 | 1.70 | 0.11 | 0 | 9 | 90 |
| 10 | TIXOSIL ® 68 | 30 | 212 | 418 | 153 | — | — | — | 20-50 | — |
| 10 | Solid formed (silica) | 138 | 253 | 396 | 976 | 0.935 | 1.157 | 2 | 9.5 | 48 |

What is claimed is:

1. Mesoporous inorganic solids:
in the form of primary and/or secondary inorganic particles of D10≧1 μm and D50≧3 μm,
wherein the size can go up to 10 mm,
of overall composition corresponding to the formula:
M_{n/q}(WₐX_{b}Y_{c}Z_{d}Oₕ)
in which M represents at least one of an ammonium ion, ions of the group IA IIA and VIIB metals, hydrogen and sodium, n and q represent respectively the equivalent fraction and the valence of the ion(s) M and n/q represents the number of moles or the molar fraction of the ion(s) M,
W represents one or more divalent elements,
X represents one or more trivalent elements,
Y represents one or more tetravalent elements,
Z represents one or more pentavalent elements,
O represents oxygen,
a, b, c and d are the respective molar fractions of W, X, Y and Z with a+b+c+d =1
1≦h≦2.5,
wherein the microporous volume (pores of size less than or equal to 2 μm) represents at most 10% of the total porous volume corresponding to the pores of size going up to 300 nm, and
A-1 wherein the mesoporous volume corresponding to the pores of size going up from 2 to 10 nm is greater than or equal to 0.18 cm³/g,
A-2 wherein the diameter of the maximum distribution peak DFT (Dmax) is such that 2 nm≦Dmax≦10 nm,
and A-3 wherein the porous volume corresponding to the pores of size Dmax±15% represents at least 70% of the porous volume corresponding to the pores of size ranging between 2 and 10 nm, or
B-1 wherein the mesoporous volume corresponding to pores going from 4 to 15 nm is greater than or equal to 0.7 cm³/g,
B-2 wherein the diameter of the maximum distribution peak DFT (Dmax) ranges between 4 and 15 nm
and B-3 wherein the porous volume corresponding to the pores of size Dmax±20% represents at least 45% of the porous volume corresponding to the pores of size ranging between 4 and 15 nm.

2. Inorganic solids according to claim 1, wherein D10≧2 μm and D50≧10 μm.

3. Inorganic solids according to claim 1, wherein the size can go up to 3 mm.

4. Inorganic solids according to claim 3, wherein the size can go up to 1.5 mm.

5. Inorganic solids according to claim 1, in which M represents at least one of a hydrogen ion and a sodium ion.

6. Inorganic solids according claim 1, wherein W represents at least one of manganese, cobalt, iron and magnesium.

7. Inorganic solids according to claim 1, wherein X represents at least one of aluminum, boron, iron and gallium.

8. Inorganic solids according to claim 1, wherein Y represents at least one of silicon and germanium.

9. Inorganic solids according to claim 8, wherein Y represents silicon.

10. Inorganic solids according to claim 1, wherein Z is phosphorus.

11. Inorganic solids according to claim 1, wherein the mesoporous volume corresponding to the pores of size going up from 2 to 10 nm is greater than or equal to 0.3 cm3/g,

12. Inorganic solids according to claim 1, wherein the diameter of the maximum distribution peak DFT (Dmax) is such that 2 nm≦Dmax≦5 nm

13. Inorganic solids according to claim 1, wherein the porous volume corresponding to the pores of size Dmax±15% represents at least 80% of the porous volume corresponding to the pores of size ranging between 2 and 10 nm.

14. Inorganic solids according to claim 13, wherein the porous volume corresponding to the pores of size Dmax±15% represents at least 90% of the porous volume corresponding to the pores of size ranging between 2 and 10 nm.

15. Inorganic solids according to claim 1, wherein the mesoporous volume corresponding to pores going from 4 to 15 nm is greater than or equal to 1 cm³/g,

16. Inorganic solids according to claim 1, wherein the porous volume corresponding to the pores of size Dmax±20% represents at least 50% of the porous volume corresponding to the pores of size ranging between 4 and 15 nm.

17. Inorganic solids according to claim 1 of the overall composition corresponding to the formula:
M n/q (X_{b}Y_{c}Oₕ)
with X=Al, Y=Si and possibly Ti, b+c=1 and 0≦b<1.

18. Inorganic solids according to claim 17, wherein Y=Si and Ti

19. Inorganic solids according to claim 1 of silica base.

20. Inorganic solids according to claim 1, wherein the proportion of fine particles of size less than or equal to 4 μm is 0%.

21. Process for the preparation of mesoporous inorganic solids according to claim 1, comprising the steps of:
placing in contact and reacting a reaction mixture comprising:
a solid inorganic source in the form of primary and/or secondary particles of D10≧1 μm and of D50≧3 μm, wherein the size can go up to 10 mm, of overall composition corresponding to the formula:
M_{n/q}(WₐX_{b}Y_{c}Z_{d}Oₕ)
where M, W, X, Y, Z, n, q, a, b, c, d and h are such as defined in claim 1,
a mobilizing agent of the solid inorganic source,
a pore calibrating agent,
and a solvent,
optionally in the presence of an inflating agent which solubilizes in the micelles,
then filtering, washing, drying and possibly eliminating the pore calibrating agent and calcination of the inorganic particles obtained, wherein the conditions of temperature, agitation and duration of the reaction are such that no appreciable modification of the morphology and of the size of the particles present during said reaction is observed.

22. Process according to claim 21, wherein said solid inorganic source is in the form of primary and/or secondary particles of D10≧2 μm and of D50≧10 μm.

23. Process according to claim 21, , wherein the pore calibrating agent is a surface active agent.

24. Process according to claim 21, wherein the solvent is water.

25. Process according to claim 21, wherein the inflating agent is trimethylbenzene,

26. Process according to claim 21, wherein the pore calibrating agent(s) are selected from the surface active agents comprising quaternary ammonium or phosphonium ions, substituted by aryl or alkyl groups having from 6 to 36 carbon atoms, which may be identical or different, in association with hydroxides, halide or silicate anions as well as amines such as dodecylamine and hexadecylamine.

27. Process according to claim 26, wherein said quaternary ammonium or phosphonium ions are cetyltrimethylammonium, cetyltrimethylphosphonium, octadecyltrimethylammonium, octadecyltrimethylphosphonium, benzyltrimethylammonium, cetylpyridinium, decyltrimthylammonium, dimethyldidodecylammonium, or trimethyldodecylammonium ions

28. Process according to claim 26, wherein said amines are dodecylamine or hexadecyl amine.

29. Process according to claim 21, wherein the solvent is organic or aqueous.

30. Process according to claim 29, wherein the solvent is aqueous.

31. Method for polymerizing non-olefinic polymers comprising reacting monomer in the presence of a catalytic component support comprising the mesoporous inorganic solids as defined in claim.

32. Method according to claim 31, wherein said mesoporous inorganic solid has D50≧10 μm.

33. Method for refining a petrochemical comprising reacting said petrochemical in the presence of a reaction catalyst comprising the mesoporous inorganic solids as defined by claim 1.

34. Method according to claim 33, wherein said process comprises at least one of alkylation, isomerization, dismutation, and cracking reactions.

35. Method for separating components of a gaseous or liquid mixture comprising at least two different compounds by adsorption functioning in a cyclic manner including alternatively functioning stages comprising the steps of:
(a) having said mixture pass in an adsorption zone containing the mesoporous inorganic solids of claim 1 and recovering either the least or less adsorbed compound(s) or a gaseous mixture enriched in the less or least adsorbed compound(s) at an exit of said adsorption zone; and
(b) desorbing the adsorbed compound(s) in the adsorption zone and regenerating the adsorption zone in a manner so as to restore to it its adsorption capacity.

36. The method according to claim 35, wherein said mesoporous inorganic solids are those of a size greater than or equal to 0.5 mm.

37. The method according to claim 36, further wherein said mesoporous inorganic solids are agglomerated with a binder.

38. Method for separating the components of a gaseous and/or liquid mixture comprising at least two different compounds comprising passing said mixture through a chromatography column containing the mesoporous inorganic solids as defined in claim 1 as supports.

39. The method according to claim 38, wherein said mesoporous inorganic solids are those wherein 1≦D10≦3 μm and 3≦D50≦15 μm.
